(19) United States
(12) Patent Application Publication
(10) Pub. No.: US 2024/0366605 A1
(43) Pub. Date: Nov. 7, 2024

(54) USE OF QUINAZOLINONE COMPOUND FOR THE TREATMENT OF CANCER

(71) Applicant: Hoffmann-La Roche Inc., Little Falls, NJ (US)

(72) Inventors: Ester BONFILL TEIXIDOR, Barcelona (ES); Raffaella IURLARO, Barcelona (ES); Piergiorgio Francesco Tommaso PETTAZZONI, Basel (CH); Juan SEOANE SUÁREZ, Barcelona (ES); Juergen WICHMANN, Steinen (DE)

(73) Assignee: Hoffmann-La Roche Inc., Little Falls, NJ (US)

(21) Appl. No.: 18/653,975

(22) Filed: May 3, 2024

Related U.S. Application Data
(63) Continuation of application No. PCT/EP2022/080475, filed on Nov. 2, 2022.

(30) Foreign Application Priority Data
Nov. 4, 2021 (EP) 21206336.6

(51) Int. Cl. A61K 31/517; A61K 9/00; A61K 9/02; A61K 9/08; A61K 9/20; A61K 9/48; A61K 31/4523; A61K 45/06; A61K 47/10; A61K 47/12; A61K 47/26; A61K 47/32; A61K 47/38

(52) U.S. Cl. CPC A61K 31/517; A61K 9/009; A61K 9/02; A61K 9/08; A61K 9/2013; A61K 9/2018; A61K 9/2054; A61K 9/4825; A61K 9/485; A61K 9/4858; A61K 9/4866; A61K 9/4875; A61K 31/4523; A61K 45/06; A61K 47/10; A61K 47/12; A61K 47/26; A61K 47/32; A61K 47/38

(57) ABSTRACT

The present invention is directed to (3R)—N—[2-cyano-4-fluoro-3-(3-methyl-4-oxo-quinazolin-6-yl)oxy-phenyl]-3-fluoro-pyrrolidine-1-sulfonamide or a pharmaceutically acceptable salt thereof, for use in the treatment of cancer, wherein the patient suffering from said cancer was previously under treatment with a different BRAF inhibitor.

### CROSS-REFERENCE TO RELATED APPLICATIONS

This application is a Continuation of International Application No. PCT/EP2022/080475, filed on Nov. 2, 2022, which claims benefit of priority to European Application No. 21206336.6 filed on Nov. 4, 2021, each of which is incorporated herein by reference in its entirety.

### BACKGROUND

Therapeutic benefits of approved BRAF inhibitors (BRAFi) as monotherapy or in combination with a MEK inhibitor (MEKi) in BRAF mutated cancer patients presenting metastasis are of fairly limited duration highlighting the need for better therapeutic options. For instance, in brain metastatic patients with BRAF V600E/K positive melanoma, the clinical benefits of available BRAFi/MEKi combinations are of far shorter duration compared to patients with metastatic sites outside of the brain and frequently relapse occurs primarily at the brain metastatic site (Lancet Oncol. 2017 July; 18(7):863-873).

Mechanisms of resistance to the three approved BRAFi (vemurafenib, dabrafenib and encorafenib), but also to their combination with MEKi (cobimetinib, trametinib and binimetinib), occurs in most cases, trough restoration of MAPK signaling which is predominantly mediated by the acquisition of genetic events enabling RAF dimerization (Cancer Discov. 2019 March; 9(3):329-341). Mechanistic studies indeed revealed that while BRAF V600E/K signal as monomeric protein, a conformation effectively inhibited by available inhibitors, in RAF dimers, only one protomer can be efficiently bound by BRAFi while the second manifest a conformation unfavourable for drug binding (Nature 2010; 464(7287):427-30 doi 10.1038/nature08902).

Multiple genetic events are described to enable RAF-dimer mediated signaling in preclinical and clinical reports, which include RAS (HRAS, KRAS, NRAS) mutations, activation of receptor tyrosine kinase (RTK) signaling, BRAF amplification and expression of the dimer forming BRAF splice variant p61. (Cancer Discov. 2014 January; 4(1):94-109 Eur J Cancer 2015; 51(18):2792-9 doi 10.1016/j.ejca.2015.08.022).

The present invention relates to the use of a novel potent, brain penetrant, paradox breaker BRAFi (Compound Ia), which was developed to better address the unmet need in metastatic cancer patients bearing a BRAF mutation such as e.g. V600E/K, in particular in brain metastatic patients.

In vivo testing of Compound Ia in primary tumors from brain metastatic lesions progressing on BRAFi monotherapy or combination therapy with a MEKi, revealed potent antitumor activity of Compound Ia providing preclinical support for the activity of this agent even after relapse to BRAFi/MEKi.

By modeling in vivo brain metastatic and peripheral lesions, Compound Ia promoted long lasting responses in both compartments and triggered dramatic responses upon relapse to available BRAFi and BRAFi/MEKi co-treatment.

Together our results reveal that Compound Ia has the potential to drive efficacy after relapse with an approved BRAFi as monotherapy or even upon relapse from approved BRAFi/MEKi combinations. In addition, the Compound Ia also has the potential to significantly prolongate the overall survival of the affected individuals when switching to a treatment with Compound Ia. Surprisingly, switching to Compound Ia after relapse with an approved BRAF inhibitor has the potential to provide for partial and in some cases even complete disease remission. In conclusion, Compound Ia has the potential to provide a new beneficial treatment option for cancer patients bearing a BRAF mutation that have previously relapsed under a BRAFi in monotherapy or MEKi combination setting, in particular in patients where brain metastasis occurred.

### SUMMARY OF THE DISCLOSURE

The present invention provides a compound of formula (I):

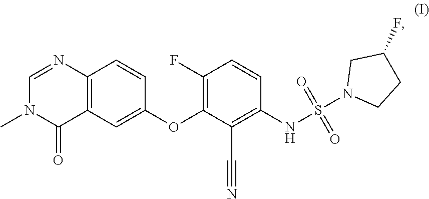 [SMILES: Cn1cnc2ccc(Oc3c(F)ccc(NS(=O)(=O)N4CC[C@@H](F)C4)c3C#N)cc2c1=O]

or a pharmaceutically acceptable salt thereof, for use in the treatment of cancer, wherein the patient suffering from said cancer was previously under treatment with a different BRAF inhibitor.

The invention further relates to novel methods and uses of the compound of formula (I) as defined above.

The chemical name of the compound of formula (I) is (3R)—N-[2-cyano-4-fluoro-3-(3-methyl-4-oxo-quinazolin-6-yl)oxy-phenyl]-3-fluoro-pyrrolidine-1-sulfonamide. Herein, the compound of formula (I) is also referred to as Compound Ia.

### BRIEF DESCRIPTION OF THE SEVERAL VIEWS OF THE DRAWINGS

FIG. 1A shows the effect of Compound Ia on tumour volume in individual mice with subcutaneously implanted A375 tumours treated with either vehicle, dabrafenib or Compound Ia. In a fourth treatment cohort, dabrafenib was administered until tumor relapse occurred, at which point dabrafenib treatment was discontinued and mice were instead treated with Compound Ia. FIG. 1B shows the effect drug treatment on probability of survival in mice with subcutaneously implanted A375 tumours. Mice were treated with either vehicle, dabrafenib or Compound Ia. In a fourth treatment cohort, dabrafenib was administered until tumor relapse occurred, at which point dabrafenib treatment was discontinued and mice were instead treated with Compound Ia.

FIG. 2A shows the effect of Compound Ia on probability of survival in mice with intracranially implanted A375 tumours. Mice were treated with either vehicle, dabrafenib or Compound Ia. In a fourth treatment cohort, dabrafenib was administered until tumor relapse occurred, at which point dabrafenib treatment was discontinued and mice were instead treated with Compound Ia.

FIG. 2B shows the effect of Compound Ia on tumour volume in individual mice with intracranially implanted A375 tumours demonstrating disease relapse under dabrafenib treatment.

FIG. 3 shows the effect of Compound Ia on probability of survival in mice with intracranially implanted A375 tumours after relapse under dabrafenib/trametinib treatment or under encorafenib/binimetinib treatment occurred. Mice were treated with either vehicle, dabrafenib/trametinib, encorafenib/binimetinib or Compound Ia. In two additional treatment cohorts, dabrafenib/trametinib and encorafenib/binimetinib were administered until tumor relapse, at which point the treatments were discontinued and mice were instead treated with Compound Ia.

### DETAILED DESCRIPTION OF THE DISCLOSURE

The term “inhibitor” denotes a compound which competes with, reduces or prevents the binding of a particular ligand to particular receptor, or which reduces or prevents the function of a particular protein. In particular, an inhibitor as used therein refers to compounds which target, decrease or inhibit activity of the respective target selected from BRAF and MEK, particular inhibitors have an IC50 value below 1 μM, below 500 nM, below 200 nM, below 100 nM, below 50 nM, below 25 nM, below 10 nM, below 5 nM, 2 nM or below 1 nM. In some embodiments of the invention the term “BRAF inhibitor” refers to compounds that decrease BRAF kinase activity at least about 10%, at least about 20%, at least about 30%, at least about 40%, at least about 50%, at least about 60%, at least about 70%, at least about 75%, at least about 80%, at least about 85%, at least about 90%, at least about 95% or at least about 99%. In some embodiments of the invention the term “MEK inhibitor” refers to compounds that decrease MEK kinase activity at least about 10%, at least about 20%, at least about 30%, at least about 40%, at least about 50%, at least about 60%, at least about 70%, at least about 75%, at least about 80%, at least about 85%, at least about 90%, at least about 95% or at least about 99%.

The term “IC50” refers to the concentration of a particular compound required to inhibit 50% of a specific measured activity.

The term “pharmaceutically acceptable salt” refers to those salts of the compound of formula (I) or of the MEK inhibitor which retain the biological effectiveness and properties of the free bases or free acids, which are not biologically or otherwise undesirable. These salts can for instance be formed with inorganic acids such as hydrochloric acid, hydrobromic acid, sulfuric acid, nitric acid, phosphoric acid and the like, in particular hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein and the like. In addition, these salts may be prepared by addition of an inorganic base or an organic base to the free acid. Salts derived from an inorganic base include, but are not limited to, the sodium, potassium, lithium, ammonium, calcium, magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyimine resins and the like. Particular pharmaceutically acceptable salts of a compound of formula (I) are the hydrochloride salts, methanesulfonic acid salts and citric acid salts. Particular pharmaceutically acceptable salts of [3,4-difluoro-2-(2-fluoro-4-iodoanilino)phenyl]-[3-hydroxy-3-[(2S)-piperidin-2-yl]azetidin-1-yl]methanone or cobimetinib are the fumarate salts and succinate salts, in particular hemifumarate salts and hemisuccinate salts.

The term “solvate” refers to non-covalent stoichiometric or nonstoichiometric combinations of solvent and solute. The term “hydrate” refers to non-covalent stoichiometric or nonstoichiometric combinations of water and solute. For example, compounds of formula (I) and pharmaceutically acceptable salts thereof, can exist in unsolvated as well as solvated forms with pharmaceutically acceptable solvents such as anisole, dichloromethane, toluene, 1,4-dioxane, water, and the like.

A certain embodiment of the invention relates to the compound of formula (I)

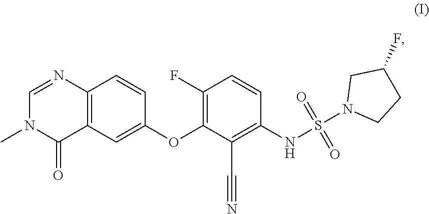 [SMILES: Cn1cnc2ccc(Oc3c(F)ccc(NS(=O)(=O)N4CC[C@@H](F)C4)c3C#N)cc2c1=O]

or a pharmaceutically acceptable salt thereof, for use in the treatment of cancer, wherein the patient suffering from said cancer was previously under treatment with a different BRAF inhibitor.

Non-limiting examples of MEK inhibitors for the use according to the invention include cobimetinib, binimetinib, trametinib, selumetinib, pimasertib, refametinib, N-[2(R),3-dihydroxypropoxy]-3,4-difluoro-2-(2-fluoro-4-iodophenylamino)benzamide (PD-325901), 2-(2-chloro-4-iodophenylamino)-N-(cyclopropylmethoxy)-3,4-difluorobenzamide (C1-1040) and 3-[2(R),3-dihydroxypropyl]-6-fluoro-5-(2-fluoro-4-iodophenylamino)-8-methylpyrido[2,3-d]pyrimidine-4,7(3H,8H)-dione (TAK-733).

In some embodiments of the present invention the MEK inhibitor is cobimetinib. Cobimetinib is an orally available, potent and highly selective inhibitor of MEK1 and MEK2, central components of the RAS/RAF pathway. Cobimethib has the chemical name [3,4-difluoro-2-(2-fluoro-4-iodoanilino)phenyl]-[3-hydroxy-3-[(2S)-piperidin-2-yl]azetidin-1-yl]methanone and has the following structure:

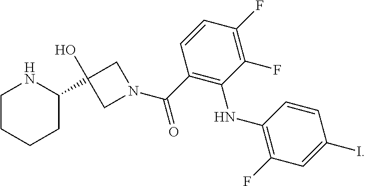 [SMILES: O=C(c1ccc(F)c(F)c1Nc1ccc(I)cc1F)N1CC(O)([C@@H]2CCCCN2)C1]

Cobimetinib may be prepared following the methods described in WO 2007/044515, which is incorporated herein by reference in its entirety. Cobimetinib is commercially available and has the following CAS Registry Number: 934660-93-2.

In some embodiments of the present invention the MEK inhibitor is binimetinib. Binimetinib is an orally available, potent and highly selective inhibitor of MEK1 and MEK2, central components of the RAS/RAF pathway. Binimetinib has the chemical name 5-[(4-bromo-2-fluorophenyl)amino]-4-fluoro-N-(2-hydroxyethoxy)-1-methyl-1H-benzimidazole-6-carboxamide and has the following structure:

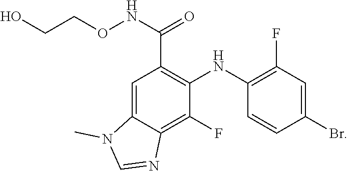 [SMILES: Cn1cnc2c(F)c(Nc3ccc(Br)cc3F)c(C(=O)NOCCO)cc21]

Binimetinib may be prepared following the methods described in WO 2003/077914, which is incorporated herein by reference in its entirety. Binimetinib is commercially available and has the following CAS Registry Number: 606143-89-9.

### Materials and Methods

### Abbreviations

DNA=deoxyribonucleic acid; PCR=polymerase chain reaction; PO=per os (latin); QD=quaque die (latin).

### Cell Line:

A375 were obtained from the American type Culture Collection (ATCC) and maintained in a humidified atmosphere with 5% CO2 at 37° C. Culture conditions are reported in the following table:

| Cell | catalog#/ | |
|---|---|---|
| line | origin | culture condition |
| A375 | CRL-1619 | Dulbecco's Modified Eagle's Medium, high glucose, |
| | | GlutaMAX (DMEM GIBCO #10566016), 10% |
| | | Fetal Bovine Serum (FBS, GIBCO #10270-106) |

### Cell Line Engineering:

A375 parental cells were transduced with virus particles of EF1a-Luciferase (firefly)-2A-GFP (Puromycin), (GenTarget Inc. cat. #LVP437-PBS). Luciferase activity was confirmed through a Luciferase Assay Reagent (Promega cat. #E1483) following the manufacturer's instructions. Viral transduction was performed for 24 h in the presence of 0.8 ug/ml Polybrene infection reagent (Millipore cat #TR-1003-G) and cells were selected with the addition of 1 ug/ml Puromycin (Thermo Fisher cat #A1113803).

### Test Agents:

Encorafenib (HY-15605), dabrafenib (HY-14660A), binimetinib (HY-15202) and trametinib (cHY-10999) were purchased from MedChemExpress, Compound Ia was internally synthesized. The compound Ia can be synthesized according to the procedures described in WO2021116055A1.

### In Vivo Experiments:

All animal experiments were approved by and performed according to the guidelines of the Institutional Animal Care Committee of the Vall d'Hebron Research Institute in agreement with the European Union and national directives. Female and male NOD scid gamma (NSG) mice of 4 to 5-week-old were purchased from Charles River.

For intracranial models, 5×10⁵ A375 luc cells were stereotactically inoculated into the corpus striatum of the right brain hemisphere (1 mm anterior and 1.8 mm lateral to the lambda; 2.5 mm intraparenchymal) of mice. Brain implanted tumors were monitored through regular Bioluminescence (BLI) signal detection and a relapse was determined upon observation of a progressive gradual increase of BLI signal.

For subcutaneous model, 5×10⁶ A375 melanoma cells were subcutaneously injected into one flank of mice. Tumour growth was monitored with a Calipher.

### EXAMPLES

The following examples and figures are provided to illustrate the invention and have no limiting character.

### Example 1

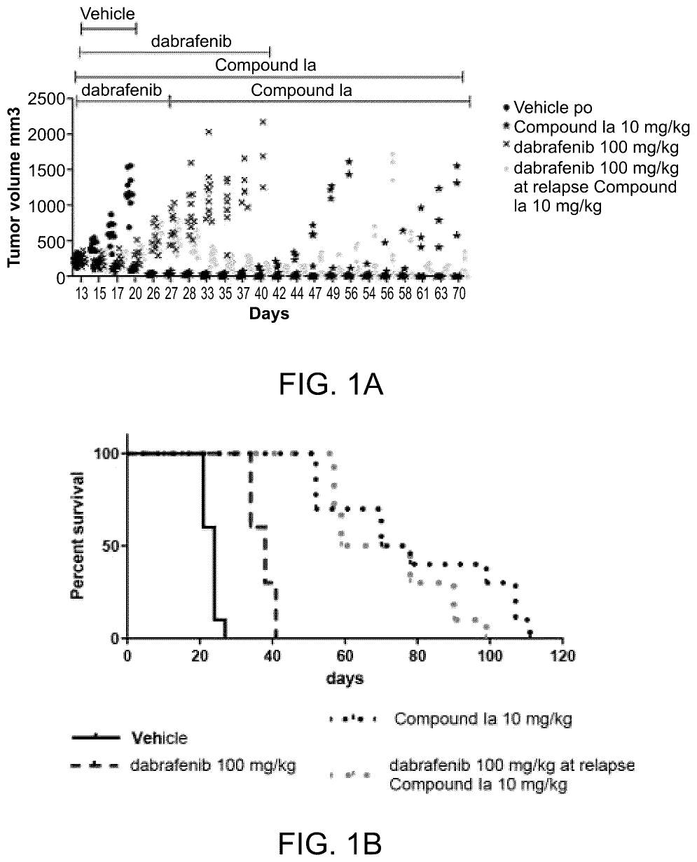

The A375 cell line presenting BRAF V600E was used as melanoma model. The cell line was stably transduced with a luciferase expressing vector to facilitate tumor size monitoring. A375 luc were implanted subcutaneously to mimic peripheral disease and then randomized when tumors reached 100-300 mm³. (10 mice/group). The mice were administered either Vehicle, Compound Ia 10 mg/kg (QD PO) or dabrafenib 100 mg/kg (QD PO). In a fourth treatment cohort, dabrafenib 100 mg/kg (QD PO) was administered until tumor relapse occurred, at which point dabrafenib treatment was discontinued and mice were instead treated with Compound Ia 10 mg/kg (QD PO). Tumour size in relapse was between 300-700 mm3, with a mean value of 558 mm³. FIG. 1A shows the effect of Compound Ia on tumour volume in individual mice with subcutaneously implanted A375 tumours after relapse under dabrafenib treatment occurred. FIG. 1B shows the effect of Compound Ia on probability of survival in mice with subcutaneously implanted A375 tumours after relapse under dabrafenib treatment occurred.

### Example 2

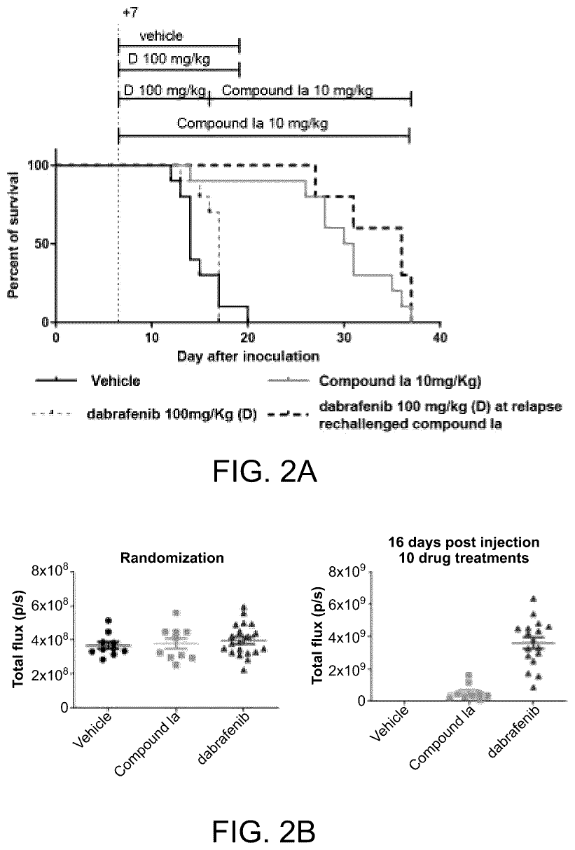

The A375 cell line presenting BRAF V600E was used as melanoma model. The cell line was stably transduced with a luciferase expressing vector to facilitate tumor size monitoring. A375 luc were implanted in the forebrain to mimic the brain metastatic setting and mice were randomized (10 mice/group). Brain implanted tumors were monitored through regular Bioluminescence (BLI) signal detection and a relapse was determined upon observation of a progressive gradual increase of BLI signal. The mice were administered either Vehicle, Compound Ia 10 mg/kg (QD PO) or dabrafenib 100 mg/kg (QD PO). In a fourth treatment cohort, dabrafenib 100 mg/kg (QD PO) was administered until tumor relapse occurred, at which point dabrafenib treatment was discontinued and mice were instead treated with Compound Ia 10 mg/kg (QD PO). FIG. 2A shows the effect of Compound Ia on probability of survival in mice with intracranially implanted A375 tumours after relapse under dabrafenib treatment occurred. FIG. 2B shows the effect of Compound Ia on tumour volume in individual mice with intracranially implanted A375 tumours after relapse under dabrafenib treatment occurred.

### Example 3

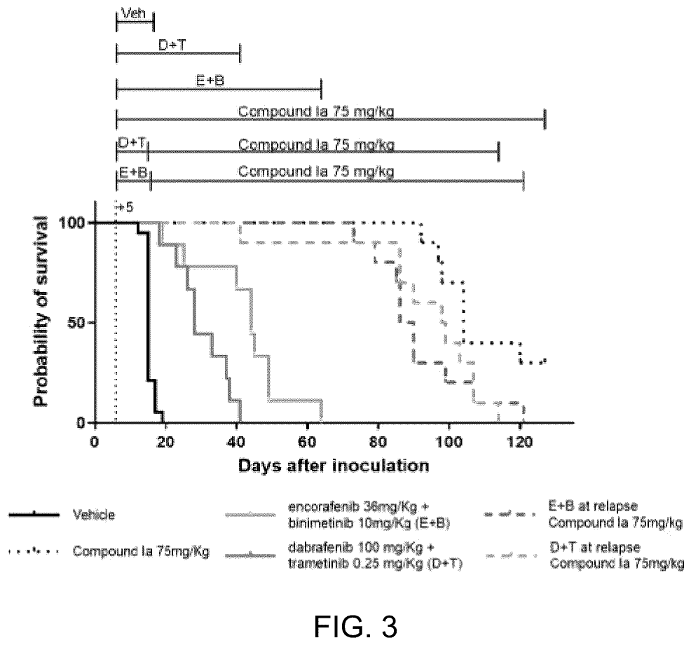

The A375 cell line presenting BRAF V600E was used as melanoma model. The cell line was stably transduced with a luciferase expressing vector to facilitate tumor size monitoring. A375 luc were implanted in the forebrain to mimic the brain metastatic setting and mice were randomized prior to treatment. Brain implanted tumors were monitored through regular Bioluminescence (BLI) signal detection and a relapse was determined upon observation of a progressive gradual increase of BLI signal (total flux (p/s) was used to estimate tumour size). Tumour size in relapse was between around 2×10⁸-5.9×10⁹ p/s, with a mean of around 2×10⁹ p/s in the group relapsing under D+T treatment. Tumour size in relapse was between around 2×10⁸-2.5×10⁹ p/s, with a mean of around 1.3×10⁹ p/s in the group relapsing under D+T treatment. FIG. 3 shows the effect of Compound Ia on probability of survival in mice with intracranially implanted A375 tumours after relapse under treatment with the approved BRAFi/MEKi combinations dabrafenib/trametinib or encorafenib/binimetinib. The mice were administered either Vehicle (19 mice), Compound Ia 75 mg/kg (10 mice), dabrafenib 100 mg/kg and trametinib 0.25 mg/kg (D+T; 19 mice), or encorafenib 36 mg/kg and binimetib 10 mg/kg (E+B; 19 mice). In a fifth cohort, cohort a combination of dabrafenib/trametinib (D+T) was administered until relapse occurred (10 mice from D+T cohort), at which point the treatment was discontinued and mice were instead treated with Compound Ia 75 mg/kg (QD PO). In a sixth cohort a combination of encorafenib/binimetinib (E+B) was administered until relapse occurred (10 mice from E+B cohort), at which point the treatment was discontinued and mice were instead treated with Compound Ia 75 mg/kg (QD PO). Administration was performed once per day and via oral route for all groups.

Described below are certain embodiments of the invention. All separate embodiments described below can be combined. Embodiments of the invention include, for example:
A compound of formula (I):

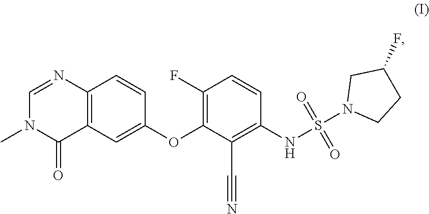 [SMILES: Cn1cnc2ccc(Oc3c(F)ccc(NS(=O)(=O)N4CC[C@@H](F)C4)c3C#N)cc2c1=O]

or a pharmaceutically acceptable salt thereof, for use in the treatment of cancer, wherein the patient suffering from said cancer was previously under treatment with a different BRAF inhibitor;
A method for the treatment or prophylaxis of cancer, which method comprises administering an effective amount of the compound of formula (I):

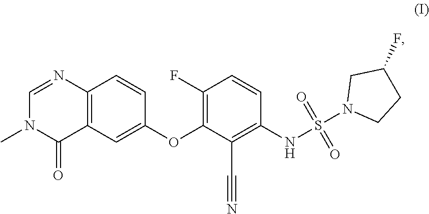 [SMILES: Cn1cnc2ccc(Oc3c(F)ccc(NS(=O)(=O)N4CC[C@@H](F)C4)c3C#N)cc2c1=O]

or a pharmaceutically acceptable salt thereof to a patient in need thereof, wherein the patient suffering from said cancer was previously under treatment with a different BRAF inhibitor;
The use of a compound of formula (I):

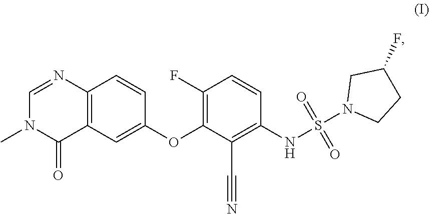 [SMILES: Cn1cnc2ccc(Oc3c(F)ccc(NS(=O)(=O)N4CC[C@@H](F)C4)c3C#N)cc2c1=O]

or a pharmaceutically acceptable salt thereof for the preparation of a medicament for the treatment or prophylaxis of cancer, wherein the patient suffering from said cancer was previously treated with a different BRAF inhibitor;
The compound for use, method or use as described herein, wherein the BRAF inhibitor of the previous treatment is selected from vemurafenib, dabrafenib and encorafenib;
The compound for use, method or use as described herein, wherein the BRAF inhibitor of the previous treatment is selected from dabrafenib and encorafenib;
The compound for use, method or use as described herein, wherein the BRAF inhibitor of the previous treatment is vemurafenib;
The compound for use, method or use as described herein, wherein the patient was previously also treated with a MEK inhibitor;
The compound for use, method or use as described herein, wherein the previously used MEK inhibitor is selected from binimetinib, trametinib and cobimetinib.
The compound for use, method or use as described herein, wherein the previously used MEK inhibitor is selected from binimetinib and trametinib;
The compound for use, method or use as described herein, wherein the previously used MEK inhibitor is cobimetinib;
The compound for use, method or use as described herein, wherein tumour relapse occurred under the previous treatment;
The compound for use, method or use as described herein, wherein the cancer is thyroid cancer, colorectal cancer, melanoma, brain cancer or non-small cell lung cancer.
The compound for use, method or use as described herein, wherein the cancer is melanoma or non-small cell lung cancer;
The compound for use, method or use as described herein, wherein the patient is suffering from brain metastases;
The compound for use, method or use as described herein, wherein the cancer is associated with BRAF^{V600} mutations;
The compound of formula (I) in combination with one or more additional anticancer agents selected from MEK inhibitors, MEK degraders, EGFR inhibitors, EGFR degraders, inhibitors of HER2 and/or HER3, degraders of HER2 and/or HER3, SHP2 inhibitors, SHP2 degraders, Axl inhibitors, Axl degraders, ALK inhibitors, ALK degraders, PI3K inhibitors, PI3K degraders, SOS1 inhibitors, SOS1 degraders, signal transduction pathway inhibitors, checkpoint inhibitors, modulators of the apoptosis pathway, cytotoxic chemotherapeutics, angiogenesis-targeted therapies, immune-targeted agents, and antibody-drug conjugates, for use as described herein;
The compound of formula (I) in combination with a MEK inhibitor for use as described herein;
A combination as described herein, wherein the MEK inhibitor is cobimetinib or a pharmaceutically acceptable salt thereof;
The method as described herein, wherein the patient is treated with one or more additional anticancer agents selected from MEK inhibitors, MEK degraders, EGFR inhibitors, EGFR degraders, inhibitors of HER2 and/or HER3, degraders of HER2 and/or HER3, SHP2 inhibitors, SHP2 degraders, Axl inhibitors, Axl degraders, ALK inhibitors, ALK degraders, PI3K inhibitors, PI3K degraders, SOS1 inhibitors, SOS1 degraders, signal transduction pathway inhibitors, checkpoint inhibitors, modulators of the apoptosis pathway, cytotoxic chemotherapeutics, angiogenesis-targeted therapies, immune-targeted agents, and antibody-drug conjugates;
The method as described herein, wherein the patient is treated with a MEK inhibitor;
The method as described herein, wherein the MEK inhibitor is cobimetinib or a pharmaceutically acceptable salt thereof;
The use of a compound of formula (I) or a pharmaceutically acceptable salt thereof as described herein, in combination with one or more additional anticancer agents selected from MEK inhibitors, MEK degraders, EGFR inhibitors, EGFR degraders, inhibitors of HER2 and/or HER3, degraders of HER2 and/or HER3, SHP2 inhibitors, SHP2 degraders, Axl inhibitors, Axl degraders, ALK inhibitors, ALK degraders, PI3K inhibitors, PI3K degraders, SOS1 inhibitors, SOS1 degraders, signal transduction pathway inhibitors, checkpoint inhibitors, modulators of the apoptosis pathway, cytotoxic chemotherapeutics, angiogenesis-targeted therapies, immune-targeted agents, and antibody-drug conjugates;
The use of a compound of formula (I) or a pharmaceutically acceptable salt thereof as described herein, in combination with a MEK inhibitor; and
The use of a compound of formula (I) or a pharmaceutically acceptable salt thereof as described herein, wherein the MEK inhibitor is cobimetinib.

Structures depicted herein are also meant to include compounds that differ only in the presence of one or more isotopically enriched atoms. Particular examples of radioisotopes are ²H, ³H, ¹³C ¹⁴C and ¹⁸F. For example the structures wherein one or more hydrogen atoms are replaced by deuterium or tritium, or one or more carbon atoms are replaced by a ¹³C- or ¹⁴C-enriched carbon are within the scope of this invention.

Furthermore, the invention includes all optical isomers, i.e. diastereoisomers, diastereomeric mixtures, racemic mixtures, all their corresponding enantiomers and/or tautomers as well as their solvates, wherever applicable, of the compound of formula (I). If desired, racemic mixtures of the compound of the invention may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography.

In the embodiments, where an optically pure enantiomer is provided, optically pure enantiomer means that the compound contains >90% of the desired isomer by weight, particularly >95% of the desired isomer by weight, or more particularly >99% of the desired isomer by weight, said weight percent based upon the total weight of the isomer of the compound. A chirally pure or chirally enriched compound may be prepared by chirally selective synthesis or by separation of enantiomers. The separation of enantiomers may be carried out on the final product or alternatively on a suitable intermediate.

In some embodiments, one of the additional anticancer agents is an EGFR inhibitor. Non-limiting examples of EGFR inhibitors include cetuximab (Erbitux®), panitumumab (Vectibix®), osimertinib (merelectinib, Tagrisso®), erlotinib (Tarceva®), gefitinib (Iressa®), necitumumab (Portrazza™), neratinib (Nerlynx®), lapatinib (Tykerb®), vandetanib (Caprelsa®) and brigatinib (Alunbrig®). Additional examples of EGFR inhibitors are known in the art. In some embodiments the EGFR inhibitor is an allosteric EGFR inhibitor.

In some embodiments, one of the additional anticancer agents is an inhibitor of HER2 and/or HER3. Non-limiting examples of HER2 and/or HER3 inhibitors include Iapatinib, canertinib, (E)-2-methoxy-N-(3-(4-(3-methyl-4-(6-methylpyridin-3-yloxy)phenylamino)quinazolin-6-yl)allyl)acetamide (GP-724714), sapitinib, 7-[[4-[(3-ethynylphenyl)amino]-7-methoxy-6-quinazolinyl]oxy]-N-hydroxy-heptanamide (CUDC-101), mubritinib, 6-[4-[(4-ethylpiperazin-1-yl)methyl]phenyl]-N-[(1R)-1-phenylethyl]-7H-pyrrolo[2,3-d]pyrimidin-4-amine (AEE788), irbinitinib (tucatinib), poziotinib, N-[4-[1-[4-(4-acetyl-1-piperazinyl)cyclohexyl]-4-amino-3-pyrazolo[3,4-d]pyrimidinyl]-2-methoxyphenyl]-1-methyl-2-indolecarboxamide (KIN001-111), 7-cyclopentyl-5-(4-phenoxyphenyl)-7H-pyrroIo[2,3-d]pyrimidin-4-ylamine (KIN001-051), 6,7-dimethoxy-N-(4-phenoxyphenyl)quinazolin-4-amine (KIN001-30), dasatinib, and bosutinib.

In some embodiments, one of the additional anticancer agents is an inhibitor of SHP2. Non-limiting examples of SHP2 inhibitors include 6-(4-amino-4-methylpiperidin-1-yl)-3-(2,3-dichlorophenyl)pyrazine-2-amine (SHP099), [3-[(3S,4S)-4-amino-3-methyl-2-oxa-8-azaspiro[4.5]decan-8-yl]-6-(2,3-dichlorophenyl)-5-methylpyrazin-2-yl]methanol (RMC-4550) RMC-4630, TNO155, and the compounds disclosed in WO 2015/107493, WO 2015/107494, WO 2015/107495, WO 2019/075265, PCT/U82019/056786 and PCT/182020/053019.

In some embodiments, one of the additional anticancer agents is a PI3K inhibitor. Non-limiting examples include buparlisib (BKM120), alpelisib (BYL719), samotolisib (LY3023414), 8-[(1R)-1-[(3,5-difluorophenyl)amino]ethyl]-N,N-dimethyl-2-(morpholin-4-yl)-4-oxo-4H-chromene-6-carboxamide (AZD8186), tenalisib (RP6530), voxtalisib hydrochloride (SAR-245409), gedatolisib (PF-05212384), panulisib (P-7170), taselisib (GDC-0032), trans-2-amino-8-[4-(2-hydroxyethoxy)cyclohexyl]-6-(6-methoxypyridin-3-yl)-4-methylpyrido[2,3-d]pyrimidin-7(8H)-one (PF-04691502), duvelisib (ABBV-954), N2-[4-oxo-4-[4-(4-oxo-8-phenyl-4H-1-benzopyran-2-yl)morpholin-4-ium-4-ylmethoxy]butyryl]-L-arginyl-glycyl-L-aspartyl-L-serine acetate (SF-1126), pictilisib (GDC-0941), 2-methyl-1-[2-methyl-3-(trifluoromethyl)benzyl]-6-(morpholin-4-yl)-1H-benzimidazole-4-carboxylic acid (GSK2636771), idelalisib (GS-1101), umbralisib tosylate (TGR-1202), pictilisib (GDC-0941), copanlisib hydrochloride (BAY 84-1236), dactolisib (BEZ-235), 1-(4-[5-[5-amino-6-(5-tert-butyl-1,3,4-oxadiazol-2-yl)pyrazin-2-yl]-1-ethyl-1-H-1,2,4-triazol-3-yl]piperidin-1-yl)-3-hydroxypropan-1-one (AZD-8835), 5-[6,6-dimethyl-4-(morpholin-4-yl)-8,9-dihydro-6H-[1,4]oxazino[4,3-e]purin-2-yl]pyrimidin-2-amine (GDC-0084) everolimus, rapamycin, perifosine, sirolimus and temsirolimus.

In some embodiments, one of the additional anticancer agents is an ALK inhibitor. Non-limiting examples include crizotinib (PF-02341066), ceritinib (LDK378), alectinib (alecensa), brigatinib (AP26113), lorlatinib (PF-6463922), ensartinib (X-396), entrectinib (RXDX-101), reprotectinib (TPX-0005), belizatinib (TSR-011), alkotinib (ZG-0418), foritinib (SAF-189), CEP-37440, TQ-B3139, PLB1003 and TPX-0131

In some embodiments, one of the additional anticancer agents is a checkpoint inhibitor. In some embodiments, the checkpoint inhibitor is a CTLA-4 inhibitor, a PD-1 inhibitor or a PD-L1 inhibitor. In some embodiments, the CTLA-4 inhibitor is ipilimumab (Yervoy®) or tremelimumab (GP-675,206). In some embodiments, the PD-1 inhibitor is selected from pembrolizumab (Keytruda®), nivolumab (Opdivo®) and RN888. In some embodiments, the PD-L1 inhibitor is selected from atezolizumab (Tecentriq®), avelumab (Bavencio®) and durvalumab (Imfinzi™). In some embodiments, the PD-L1 inhibitor is atezolizumab (Tecentriq®).

In some embodiments, one of the additional anticancer agents is an antibody-drug conjugate. Non-limiting examples of an antibody-drug conjugate include gemtuzumab ozogamicin (Mylotarg™), inotuzumab ozogamicin (Besponsa®), brentuximab vedotin (Adcetris®), ado-trastuzumab emtansine (TDM-f, Kadcyla®), mirvetuximab soravtansine (IMGN853) and anetumab ravtansine.

In some embodiments, one of the additional anticancer agents is an antibody such as bevacizumab (Mvasti™, Avastin®), trastuzumab (Herceptin®), avelumab (Bavencio®), rituximab (MabThera™, Rituxan®), edrecolomab (Panorex), daratumuab (Darzalex®), olaratumab (Lartruvo™), ofatumumab (Arzerra®), alemtuzumab (Campath®), cetuximab (Erbitux®), oregovomab, pembrolizumab (Keytruda®), dinutiximab (Unituxin®), obinutuzumab (Gazyva®), tremelimumab (GP-675,206), ramucirumab (Cyramza®), ublituximab (TG-1101), panitumumab (Vectibix®), elotuzumab (EmplicitiT‘V’), necitumumab (PortrazzaT‘V’), cirmtuzumab (UC-961), ibritumomab (Zevalin®), isatuximab (SAR650984), nimotuzumab, fresolimumab (GC1008), Iirilumab (INN), mogamulizumab (Poteligeo®), ficlatuzumab (AV-299), denosumab (Xgeva®), ganitumab, urelumab, pidilizumab, amatuximab, blinatumomab (AMG103; Blincyto®) or midostaurin (Rydapt).

Another embodiment of the invention provides a pharmaceutical composition containing one or more compositions, wherein each composition contains one or more compounds for use according to the invention and one or more therapeutically inert carriers, diluents or excipients, as well as a method to prepare such a pharmaceutical compositions. In one example, the compound of formula (I) may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula (I) is formulated in an acetate buffer, at pH 5. In another embodiment, the compound of formula (I) is sterile. The compound may be stored, for example, as a solid or amorphous composition, as a lyophilized formulation or as an aqueous solution

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

As used herein, a “pharmaceutically acceptable carrier” or “pharmaceutically acceptable excipient” is intended to include any and all material compatible with pharmaceutical administration including solvents, dispersion media, coatings, antibacterial and antifungal agents, isotonic and absorption delaying agents, and other materials and compounds compatible with pharmaceutical administration. Except insofar as any conventional media or agent is incompatible with the active compound, use thereof in the compositions of the invention is contemplated. Supplementary active compounds can also be incorporated into the compositions.

Pharmaceutical compositions can be obtained by processing the compound of formula (I) as described herein with pharmaceutically acceptable, inorganic or organic carriers or excipients. Lactose, corn starch or derivatives thereof, talc, stearic acids or it's salts and the like can be used, for example, as such carriers for tablets, coated tablets, dragées and hard gelatine capsules. Suitable carriers for soft gelatine capsules are, for example, vegetable oils, waxes, fats, semi-solid and liquid polyols and the like. Depending on the nature of the active substance no carriers are, however, usually required in the case of soft gelatine capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

The pharmaceutical compositions can, moreover, contain preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

Pharmaceutical compositions of the compound of formula (I), alone or in combination with a second anticancer agent, can be prepared for storage by mixing the active ingredients having the desired degree of purity with optional pharmaceutically acceptable carriers, excipients or stabilizers (Remington's Pharmaceutical Sciences 16th edition, Osol, A. (ed.) (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Pharmaceutical compositions of the compound of formula (I) include those suitable for oral, nasal, topical (including buccal and sublingual), rectal, vaginal and/or parenteral administration. The compositions may conveniently be presented in unit dosage form and may be prepared by any methods well known in the art of pharmacy. The amount of active ingredient which can be combined with a carrier material to produce a single dosage form will vary depending upon the host being treated, as well as the particular mode of administration. The amount of active ingredient which can be combined with a carrier material to produce a single dosage form will generally be that amount of the compound of formula (I) which produces a therapeutic effect. Generally, out of one hundred percent, this amount will range from about 1 percent to about 90 percent of active ingredient, preferably from about 5 percent to about 70 percent, most preferably from about 10 percent to about 30 percent. Methods of preparing these compositions include the step of bringing into association the compound of formula (I) with the carrier and, optionally, one or more accessory ingredients. In general, the pharmaceutical compositions can be prepared by uniformly and intimately bringing into association the compound of formula (I) with liquid carriers, or finely divided solid carriers, or both, and then, if necessary, shaping the product. Pharmaceutical compositions suitable for oral administration may be in the form of capsules, cachets, sachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of the compound of formula (I) as an active ingredient.

In further embodiments of the invention, the compound of formula (I) and a MEK inhibitor are formulated into one or two separate pharmaceutical compositions.

The active ingredients may also be entrapped in microcapsules prepared, for example, by coacervation techniques or by interracial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences, 16th edition, Osol, A. (ed.) (1980).

The formulations to be used for in vivo administration must be sterile. This can be readily accomplished by filtration through sterile filtration membranes.

The dosage can vary within wide limits and will, of course, have to be adjusted to the individual requirements in each particular case. In the case of oral administration the dosage for adults can vary from about 0.01 mg to about 8000 mg per day of a compound of general formula (I) or of the corresponding amount of a pharmaceutically acceptable solvate thereof. The daily dosage may be administered as single dose or in divided doses and, in addition, the upper limit can also be exceeded when this is found to be indicated. In the case of oral administration the administration can be after a high-fat meal or following a 10-hour fast.

The following examples illustrate the present invention without limiting it, but serve merely as representative thereof. The pharmaceutical compositions conveniently contain about 1-500 mg, particularly 5-250 mg, of a compound of formula (I). In certain embodiments the pharmaceutical compositions containing a compound of formula (I) contains in addition about 1-500 mg, particularly 5-80 mg, of a MEK inhibitor in a fixed-dose combination.

Non-limiting examples of compositions according to the invention are:

### Example A-1

### Tablets of the Following Composition are Manufactured in the Usual Manner:

TABLE 3
possible tablet composition

mg/tablet

| ingredient | 5 | 25 | 100 | 500 |
|---|---|---|---|---|
| 1. Compound of formula (I) | 5 | 25 | 100 | 500 |
| 2. Lactose Anhydrous DTG | 125 | 105 | 30 | 150 |
| 3. Sta-Rx 1500 | 6 | 6 | 6 | 60 |
| 4. Microcrystalline Cellulose | 30 | 30 | 30 | 450 |
| 5. Magnesium Stearate | 1 | 1 | 1 | 1 |
| Total | 167 | 167 | 167 | 831 |

### Manufacturing Procedure

1. Mix ingredients 1, 2, 3 and 4 and granulate with purified water.
2. Dry the granules at 50° C.
3. Pass the granules through suitable milling equipment.
4. Add ingredient 5 and mix for three minutes; compress on a suitable press.

### Example B-1

### Capsules of the Following Composition are Manufactured:

TABLE 4
possible capsule ingredient composition

mg/capsule

| ingredient | 5 | 25 | 100 | 500 |
|---|---|---|---|---|
| 1. Compound of formula (I) | 5 | 25 | 100 | 500 |
| 2. Hydrous Lactose | 159 | 123 | 148 | — |
| 3. Corn Starch | 25 | 35 | 40 | 70 |
| 4. Talk | 10 | 15 | 10 | 25 |
| 5. Magnesium Stearate | 1 | 2 | 2 | 5 |
| Total | 200 | 200 | 300 | 600 |

### Manufacturing Procedure

1. Mix ingredients 1, 2 and 3 in a suitable mixer for 30 minutes.
2. Add ingredients 4 and 5 and mix for 3 minutes.
3. Fill into a suitable capsule.

The compound of formula (I), lactose and corn starch are firstly mixed in a mixer and then in a comminuting machine. The mixture is returned to the mixer; the talc is added thereto and mixed thoroughly. The mixture is filled by machine into suitable capsules, e.g. hard gelatin capsules.

### Example B-2

Soft Gelatin Capsules of the following composition are manufactured:

TABLE 5
possible soft gelatin capsule ingredient composition

| ingredient | mg/capsule |
|---|---|
| Compound of formula (I) | 5 |
| Yellow wax | 8 |
| Hydrogenated Soya bean oil | 8 |
| Partially hydrogenated plant oils | 34 |
| Soya bean oil | 110 |
| Total | 165 |

TABLE 6
possible soft gelatin capsule composition

| ingredient | mg/capsule |
|---|---|
| Gelatin | 75 |
| Glycerol 85% | 32 |
| Karion 83 | 8 (dry matter) |
| Titan dioxide | 0.4 |
| Iron oxide yellow | 1.1 |
| Total | 116.5 |

### Manufacturing Procedure

The compound of formula (I) is dissolved in a warm melting of the other ingredients and the mixture is filled into soft gelatin capsules of appropriate size. The filled soft gelatin capsules are treated according to the usual procedures.

### Example C

### Suppositories of the Following Composition are Manufactured:

TABLE 7
possible suppository composition

| ingredient | mg/supp. |
|---|---|
| Compound of formula (I) | 15 |
| Suppository mass | 1285 |
| Total | 1300 |

### Manufacturing Procedure

The suppository mass is melted in a glass or steel vessel, mixed thoroughly and cooled to 45° C. Thereupon, the finely powdered compound of formula (I) is added thereto and stirred until it has dispersed completely. The mixture is poured into suppository moulds of suitable size, left to cool; the suppositories are then removed from the moulds and packed individually in wax paper or metal foil.

### Example D

### Injection Solutions of the Following Composition are Manufactured:

TABLE 8
possible injection solution composition

| ingredient | mg/injection solution. |
|---|---|
| Compound of formula (I) | 3 |
| Polyethylene Glycol 400 | 150 |
| acetic acid | q.s. ad pH 5.0 |
| water for injection solutions | ad 1.0 ml |

The compound of formula (I) is dissolved in a mixture of Polyethylene Glycol 400 and water for injection (part). The pH is adjusted to 5.0 by acetic acid. The volume is adjusted to 1.0 ml by addition of the residual amount of water. The solution is filtered, filled into vials using an appropriate overage and sterilized.

### Example E

### Sachets of the Following Composition are Manufactured:

TABLE 9
possible sachet composition

| ingredient | mg/sachet |
|---|---|
| Compound of formula (I) | 50 |
| Lactose, fine powder | 1015 |
| Microcrystalline cellulose (AVICEL PH 102) | 1400 |
| Sodium carboxymethyl cellulose | 14 |
| Polyvinylpyrrolidon K 30 | 10 |
| Magnesium stearate | 10 |
| Flavoring additives | 1 |
| Total | 2500 |

### Manufacturing Procedure

The compound of formula (I) is mixed with lactose, microcrystalline cellulose and sodium carboxymethyl cellulose and granulated with a mixture of polyvinylpyrrolidone in water. The granulate is mixed with magnesium stearate and the flavoring additives and filled into sachets.

### Specific Numbered Embodiments

All separate embodiments described below can be combined.

1. A compound of formula (I):

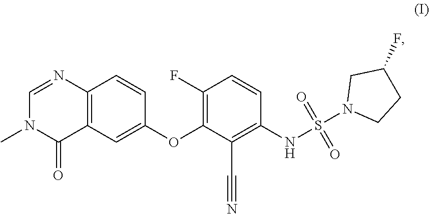 [SMILES: Cn1cnc2ccc(Oc3c(F)ccc(NS(=O)(=O)N4CC[C@@H](F)C4)c3C#N)cc2c1=O]

or a pharmaceutically acceptable salt thereof, for use in the treatment of cancer, wherein the patient suffering from said cancer was previously under treatment with a BRAF inhibitor other than the compound of formula (I).
2. A compound of formula (I):

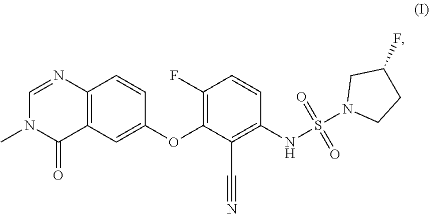 [SMILES: Cn1cnc2ccc(Oc3c(F)ccc(NS(=O)(=O)N4CC[C@@H](F)C4)c3C#N)cc2c1=O]

or a pharmaceutically acceptable salt thereof, for use in the treatment of relapsed cancer patients who have been previously treated with a BRAF inhibitor other than compound of formula (I).
3. A compound of formula (I):

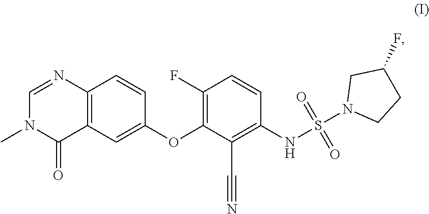 [SMILES: Cn1cnc2ccc(Oc3c(F)ccc(NS(=O)(=O)N4CC[C@@H](F)C4)c3C#N)cc2c1=O]

or a pharmaceutically acceptable salt thereof, for preventing formation of treatment resistance, in particular wherein the formation has been observed in patients having been treated with a BRAF inhibitor other than the compound of formula (I).
4. A compound of formula (I):

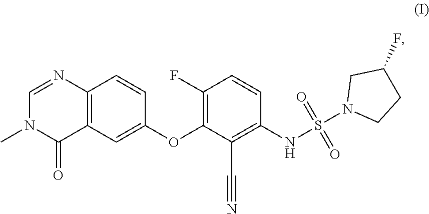 [SMILES: Cn1cnc2ccc(Oc3c(F)ccc(NS(=O)(=O)N4CC[C@@H](F)C4)c3C#N)cc2c1=O]

or a pharmaceutically acceptable salt thereof, for use in reducing treatment resistance of a cancer.
5. A compound of formula (I):

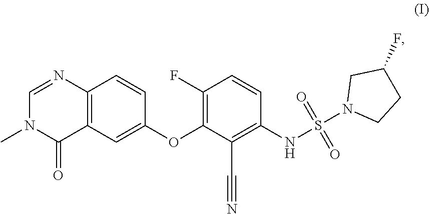 [SMILES: Cn1cnc2ccc(Oc3c(F)ccc(NS(=O)(=O)N4CC[C@@H](F)C4)c3C#N)cc2c1=O]

or a pharmaceutically acceptable salt thereof, for use in treating relapsing cancer with a BRAF mutation.
6. A method for the treatment or prophylaxis of cancer, which method comprises administering an effective amount of the compound of formula (I):

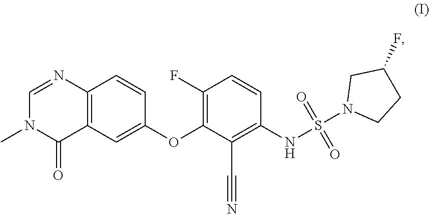 [SMILES: Cn1cnc2ccc(Oc3c(F)ccc(NS(=O)(=O)N4CC[C@@H](F)C4)c3C#N)cc2c1=O]

or a pharmaceutically acceptable salt thereof to a patient in need thereof, wherein the patient suffering from said cancer was previously under treatment with a different BRAF inhibitor.
7. A method for reducing relapse growth of a cancer cell comprising administering a therapeutically effective amount of compound of formula (I), in particular wherein said administration reduced relapse growth of the cancer cell, more particularly wherein the relapse has been observed after prior administration of at least one BRAF inhibitor other than compound of formula (I).
8. The use of a compound of formula (I):

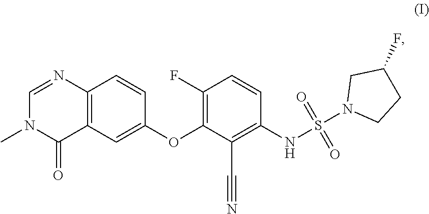 [SMILES: Cn1cnc2ccc(Oc3c(F)ccc(NS(=O)(=O)N4CC[C@@H](F)C4)c3C#N)cc2c1=O]

or a pharmaceutically acceptable salt thereof for the preparation of a medicament for the treatment or prophylaxis of cancer, wherein the patient suffering from said cancer was previously treated with a different BRAF inhibitor.
9. The compound for use, method or use according to any one of embodiments 1 to 8, wherein the BRAF inhibitor of the previous treatment is selected from vemurafenib, dabrafenib and encorafenib.
10. The compound for use, method or use according to any one of embodiments 1 to 9, wherein the BRAF inhibitor of the previous treatment is dabrafenib.
11. The compound for use, method or use according to any one of embodiments 1 to 9, wherein the BRAF inhibitor of the previous treatment is vemurafenib.
12. The compound for use, method or use according to any one of embodiments 1 to 9, wherein the BRAF inhibitor of the previous treatment is encorafenib.
13. The compound for use, method or use according to any one of embodiments 1 to 12, wherein the patient was previously also treated with a MEK inhibitor.
14. The compound for use, method or use according to any one of embodiments 1 to 13, wherein the previously used MEK inhibitor is selected from binimetinib, trametinib and cobimetinib.
15. The compound for use, method or use according to any one of embodiments 1 to 14, wherein the previously used MEK inhibitor is binimetinib.
16. The compound for use, method or use according to any one of embodiments 1 to 14, wherein the previously used MEK inhibitor is trametinib.
17. The compound for use, method or use according to any one of embodiments 1 to 14, wherein the previously used MEK inhibitor is cobimetinib.
18. The compound for use, method or use according to any one of embodiments 1 to 17, wherein tumour relapse occurred under the previous treatment.
19. The compound for use, method or use according any one of embodiments 1 to 18, wherein the cancer is thyroid cancer, colorectal cancer, melanoma, brain cancer or non-small cell lung cancer.
20. The compound for use, method or use according any one of embodiments 1 to 19, wherein the cancer is melanoma or non-small cell lung cancer.
21. The compound for use, method or use according any one of embodiments 1 to 19, wherein the cancer is melanoma or non-small cell lung cancer.
22. The compound for use, method or use according any one of embodiments 1 to 19, wherein the cancer is non-small cell lung cancer.
23. The compound for use, method or use according to any one of embodiments 1 to 22, wherein the patient is suffering from brain metastases.
24. The compound for use, method or use according to any one of embodiments 1 to 23, wherein the cancer is associated with BRAF^{V600} mutations.
25. The compound for use, method or use according to any one of embodiments 1 to 24, wherein the cancer is associated with BRAF^{V600}E mutations.
26. The compound for use, method or use according to any one of embodiments 1 to 24, wherein the cancer is associated with BRAF^{V600K} mutations.
27. The compound of formula (I) in combination with one or more additional anticancer agents selected from MEK inhibitors, MEK degraders, EGFR inhibitors, EGFR degraders, inhibitors of HER2 and/or HER3, degraders of HER2 and/or HER3, SHP2 inhibitors, SHP2 degraders, Axl inhibitors, Axl degraders, ALK inhibitors, ALK degraders, PI3K inhibitors, PI3K degraders, SOS1 inhibitors, SOS1 degraders, signal transduction pathway inhibitors, checkpoint inhibitors, modulators of the apoptosis pathway, cytotoxic chemotherapeutics, angiogenesis-targeted therapies, immune-targeted agents, and antibody-drug conjugates, for use according to any one of embodiments 1-5 or 9-26.
28. The compound of formula (I) in combination with a MEK inhibitor for use according to any one of embodiments 1-5 or 9-26.
29. A combination according to embodiments 27 or 28, wherein the MEK inhibitor is selected from binimetinib, trametinib and cobimetinib or a pharmaceutically acceptable salt thereof.
30. A combination according to embodiments 27 or 28, wherein the MEK inhibitor is cobimetinib or a pharmaceutically acceptable salt thereof.
31. The method according to any one of embodiments 6, 7 or 9-26, wherein the patient is treated with one or more additional anticancer agents selected from MEK inhibitors, MEK degraders, EGFR inhibitors, EGFR degraders, inhibitors of HER2 and/or HER3, degraders of HER2 and/or HER3, SHP2 inhibitors, SHP2 degraders, Axl inhibitors, Axl degraders, ALK inhibitors, ALK degraders, PI3K inhibitors, PI3K degraders, SOS1 inhibitors, SOS1 degraders, signal transduction pathway inhibitors, checkpoint inhibitors, modulators of the apoptosis pathway, cytotoxic chemotherapeutics, angiogenesis-targeted therapies, immune-targeted agents, and antibody-drug conjugates.
32. The method according to any one of embodiments 6, 7 or 9-26, wherein the patient is treated with a MEK inhibitor.
33. The method according to any one of embodiments 31 or 32, wherein the MEK inhibitor is selected from binimetinib, trametinib and cobimetinib or a pharmaceutically acceptable salt thereof.
34. The method according to any one of embodiments 31-33, wherein the MEK inhibitor is cobimetinib or a pharmaceutically acceptable salt thereof.
35. The method according to any one of embodiments 6, 7 or 9-26, wherein the patient is treated with a checkpoint inhibitor.
36. The method according to embodiment 35, wherein the checkpoint inhibitor is atezolizumab (Tecentriq®).
37. The use of a compound of formula (I) or a pharmaceutically acceptable salt thereof according to any one of embodiments 8 to 26, in combination with one or more additional anticancer agents selected from MEK inhibitors, MEK degraders, EGFR inhibitors, EGFR degraders, inhibitors of HER2 and/or HER3, degraders of HER2 and/or HER3, SHP2 inhibitors, SHP2 degraders, Axl inhibitors, Axl degraders, ALK inhibitors, ALK degraders, PI3K inhibitors, PI3K degraders, SOS1 inhibitors, SOS1 degraders, signal transduction pathway inhibitors, checkpoint inhibitors, modulators of the apoptosis pathway, cytotoxic chemotherapeutics, angiogenesis-targeted therapies, immune-targeted agents, and antibody-drug conjugates.
38. The use of a compound of formula (I) or a pharmaceutically acceptable salt thereof according to any one of embodiments 8 to 26, in combination with a MEK inhibitor.
39. The use of a compound of formula (I) or a pharmaceutically acceptable salt thereof according to any one of embodiments 37 or 38, wherein the MEK inhibitor is cobimetinib, or a pharmaceutically acceptable salt thereof.

Although the foregoing invention has been described in some detail by way of illustration and example for purposes of clarity of understanding, the descriptions and examples should not be construed as limiting the scope of the invention. The disclosures of all patent, patent applications and scientific literature cited herein are expressly incorporated in their entirety by reference.

1. A method of treating a cancer, the method comprising administering to a patient suffering from the cancer a compound of formula (I): 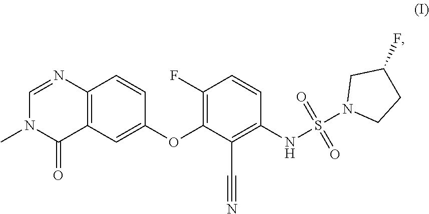 [SMILES: Cn1cnc2ccc(Oc3c(F)ccc(NS(=O)(=O)N4CC[C@@H](F)C4)c3C#N)cc2c1=O]
or a pharmaceutically acceptable salt thereof, wherein the patient suffering from the cancer was previously under treatment with a BRAF inhibitor other than the compound of formula (I).

2. The method of claim 1, wherein the BRAF inhibitor of the previous treatment is selected from vemurafenib, dabrafenib and encorafenib, or a pharmaceutically acceptable salt thereof.

3. The method of claim 2, wherein the patient was previously also treated with a MEK inhibitor.

4. The method of claim 3, wherein the previously used MEK inhibitor is selected from binimetinib, trametinib and cobimetinib, or a pharmaceutically acceptable salt thereof.

5. The method of claim 1, wherein a tumor relapse occurred under the previous treatment.

6. The method of claim 1, wherein the cancer is thyroid cancer, colorectal cancer, melanoma, brain cancer or non-small cell lung cancer.

7. The method of claim 6, wherein the cancer is melanoma or non-small cell lung cancer.

8. The method of claim 1, wherein the patient is suffering from brain metastases.

9. The method of claim 1, wherein the cancer is associated with BRAF^{V600} mutations.

10. The method of claim 1, wherein the compound of formula (I) is administered to the patient in combination with one or more additional anticancer agents selected from MEK inhibitors, MEK degraders, EGFR inhibitors, EGFR degraders, inhibitors of HER2 and/or HER3, degraders of HER2 and/or HER3, SHP2 inhibitors, SHP2 degraders, Axl inhibitors, Axl degraders, ALK inhibitors, ALK degraders, PI3K inhibitors, PI3K degraders, SOS1 inhibitors, SOS1 degraders, signal transduction pathway inhibitors, checkpoint inhibitors, modulators of the apoptosis pathway, cytotoxic chemotherapeutics, angiogenesis-targeted therapies, immune-targeted agents, and antibody-drug conjugates.

11. The method of claim 1, wherein the compound of formula (I) is administered to the patient in combination with a MEK inhibitor.

12. The method of claim 11, wherein the MEK inhibitor is cobimetinib, or a pharmaceutically acceptable salt thereof.

13-17. (canceled)
